(19) United States
(12) Patent Application Publication
(10) Pub. No.: US 2002/0016490 A1
(43) Pub. Date: Feb. 7, 2002

(54) Granules of azo-type compounds

(76) Inventors: Jean-Yves Dauba, Lescar (FR); Alexandre Goldszal, Pau (FR)

Correspondence Address: PENNIE & EDMONDS LLP, 1155 Avenue of the Americas, New York, NY, 10036-2711, US

(21) Appl. No.: 09/837,064

(22) Filed: Apr. 18, 2001

Related U.S. Application Data
(63) Continuation of application No. PCT/FR99/02249, filed on Sep. 22, 1999.

(30) Foreign Application Priority Data
Oct. 23, 1998 (FR) 98/13325

(51) Int. Cl. C07C 255/00

(52) U.S. Cl. 558/303

(57) ABSTRACT

A composition, comprising an azo compound in the form of granules having a diameter size of between 200 μm and 10 mm, the cumulative pore volume of said granules being between about 0.2 and about 2 ml per gram of composition. A process for preparing the composition is also provided, which comprises providing a wetted powder comprising the azo compound, effecting granulation by passing the wetted powder under mechanical stress, through a die, and then drying the granules so obtained at a temperature of at or less than about 45° C.

### FIELD OF THE INVENTION

The present invention relates to a composition, comprising at least one azo-type compound in the form of granules, as well as a process for preparing the said composition.

### BACKGROUND OF THE INVENTION

Azo-type compounds, especially 2,2′- azobis (isobutyronitrile), are used as initiators for polymerization reactions involving free radicals.

These reactions may be bulk, solution, suspension or emulsion polymerization reactions of various monomers, for example (meth)acrylic or vinyl monomers such as acrylamide, acrylonitrile, alkyl (meth)acrylate, styrene, vinyl acetate, vinyl chloride and xrinylidene chloride. The ranges of application are varied and relate especially (but not exclusively) to acrylic sheets or fibres, flocculants, paints, coating resins, grafted polyols, polystyrene, PVC, PVA and PMMA.

Azo compounds, and in particular 2,2′-azobis (isobutyronitrile), are generally made available in powder form by draining the suspension resulting from the final step of the synthesis. The particles of this powder have a size of between 3 μm and 150 μm, preferably between 10 μm and 150 μm. Having the azo compounds in powder form makes it possible to obtain very rapidly a homogeneous distribution of the compound throughout the mass of a solvent or water. Such a rapid distribution is highly desirable, especially when the azo compounds are used in industrial polymerization plants. Depending on the applications envisaged, and for the first step in their production, the azo compounds are in fact either in suspension in water or dissolved in organic solvents, such as dichloromethane, methyl ethyl ketone, acetone, chloroform, ethyl acetate and toluene.

In some industrial polymerization plants, automatic devices for introducing (or feeding) the reactants into the reactors allow for improved production automation. However, the presentation of the azo-type initiators in powder form, as described above, does not make it possible to have a pourable product lending itself to such processing.

Attempts have been made to remedy this drawback by proposing presentations in the form of pourable powders which are generally prepared by incorporating a flow agent such as silica, into the above powder.

However, these powders, if released into the atmosphere in the form of a dust, are liable to come into contact with the upper respiratory tracts of persons handling them. Such contact may occur with operators present on the industrial site, especially when manually loading the reactors with the powder or who carry out the sampling necessary for controlling the manufacturing process. It is desirable to minimize, or even completely prevent, any contact of this type in the case of azo compounds which may pose a toxic risk, in order to ensure the safety of operators and guarantee the hygiene of the manufacturing areas.

Unfortunately, the presentation of the azo compounds in the form of a pourable powder generates even more inhalable particles than powders without a flow agent.

### SUMMARY OF THE INVENTION

One object of the present invention is to remedy the foregoing drawbacks.

Another object of the invention is to provide a composition of an azo compound which allows the compound to be homogeneously and rapidly distributed when in use.

Another object of the invention is to provide a solid composition of an azo compound that can flow in the manner of a liquid.

Another object of the invention is to provide a solid composition of an azo compound free of fine particles or dust that can come into contact with workers, and especially with the human respiratory system.

It has now been found that these objects can be completely or partly achieved by means of the compositions and methods according to the present invention.

An intendment of the present invention is a composition comprising at least one azo compound of formula (I):

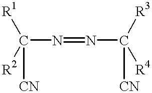

wherein:

R¹, R², R³ and R⁴ can be the same or different such that each independently represents:

an alkyl group, preferably a C₁-C₆-alkyl group, optionally having one or more hydroxyl, alkoxy, carboxyl or halogen substituents, or

a cycloalkyl group, preferably having from 3 to 6 carbon atoms, optionally having one or more hydroxyl, alkoxy, carboxyl or halogen substituents;

an aryl group such as phenyl or naphthyl, optionally having one or more hydroxyl, alkyl, alkoxy, carboxyl or halogen substituents, or

an aralkyl group, such as benzyl or phenethyl, optionally having one or more alkyl, alkoxy, hydroxyl, carboxyl or halogen substituents, or

at least one of the combinations of R¹ with R² and of R³ with R⁴forms a cycloalkyl radical.

The composition is further characterized in that the azo compound is in the form of granules having a size of between 200 μm and 10 mm, preferably between 250 μm and 5 mm, the cumulative pore volume of said granules being between 0.2 and 2 ml and preferably between 0.5 and 1.3 ml per gram of composition.

The compounds of formula (I) can be prepared using known processes.

The invention also relates to a process for preparing the granules according to the invention. This process comprises, in succession:

(i) wetting the powder obtained by draining the suspension resulting from the final step of the process for synthesizing the azo compound, so as to obtain a powder comprising roughly from 10 to 45%, preferably about 15 to about 40%, of water;

(ii) granulating the wetted powder by passing said powder, under mechanical stress, through a die, the thickness e and the diameter of the perforations d of which are such that the e/d ratio is roughly between 0.5 and 3, preferably between about 0.5 and about 2, the diameter d being close to the desired size of the granules and the stress being such that the temperature of the powder remains less than 40° C., preferably less than 30° C.;

(iii) drying of the granules at a temperature of below about 45° C., preferably at or about 35° C.

In step (i), the percentage of water in the powder corresponds to the weight ratio of water to wet powder.

Advantageously, the process provides a composition in the form of granules according to the invention, while preventing, during the said process, any onset of decomposition of the azo compound. Such decomposition may in fact lead to enhanced and spontaneously accelerated decomposition of the product, giving rise to the release of toxic and inflammable gas. The absence of any risk of such a decomposition is a very important advantage of the process according to the invention.

In step (ii) of the process according to the invention, passing the wetted powder through the die is carried out, for example, by a granulating press, in which the powder is pushed against the die by means of rotating blades.

### DETAILED DESCRIPTION OF THE INVENTION

Within description of the present invention, all percentages given are by weight unless otherwise indicated.

As used herein, the expression “inhalable particles” means particles having a size of less than 20 μm, preferably less than 5 μm, which are capable of appearing throughout the handling of the granules as defined below, especially when they are flowing, and which, because of their size, can, being entrained by the air, penetrate workers' lungs.

The content of inhalable particles is measured using the method explained below.

The principle of the method consists in dropping a known quantity of granules into a closed measurement chamber, so as to expose the inhalable particles and then collecting them on a filter by means of a suction device, and thus to determine the corresponding mass. A microporous filter placed on the filter holder of a filtration device is weighed. The filtration device is connected to a flow meter and to a vacuum pump which is controlled so that the air flow rate is set to 15 litres per minute. Thirty (30) g of granules to be analyzed are poured into the top of the tube of the measurement apparatus, which tube, 60 cm in height and 4 cm in diameter, terminates in a closed chamber in the form of a cube having edges of 20 cm. The filtration device is placed on one side face of this chamber. The air containing the inhalable particles liberated during the fall of the granules is extracted for 60 seconds and the particles are thus collected on the filter. The filter is recovered and weighed. The content of inhalable particles of the granules analyzed, expressed in milligrams, is represented by the difference in weight of the filter before and after the analysis.

By way of indication, a product having a content of inhalable particles of:

3 to 10 mg is termed “slightly dusty”;

30 mg is termed “dusty”;

200 mg is termed “very dusty”.

The pourability is measured using the method explained below.

Seven funnels, numbered from 1 to 7, are used, these having a height of 60 mm and the following upper and lower diameter characteristics:

| | Upper diameter | |
|---|---|---|
| Funnel No. | (mm) | Lower diameter |
| 1 | 80 | 29.4 |
| 2 | 75 | 25.0 |
| 3 | 69 | 20.5 |
| 4 | 64 | 16.6 |
| 5 | 60 | 9.9 |
| 6 | 53.5 | 4.9 |
| 7 | 52.5 | 3.0 |

These seven funnels are each placed vertically by means of a suitable device and aligned on a horizontal work surface in the order of increasing funnel number. The method consists in dropping a known quantity of granules in each funnel of the series, starting from Number 1, and in repeating the operation for each funnel of the series in the order of increasing funnel number.

The flowability is represented by the number of the last funnel of the series in which the granules flow. The flowability is therefore better the higher the funnel number. Thus, by way of illustration, a flowability of 3 corresponds to a non-pourable product. A flowability of 4 to 7 corresponds to satisfactory behaviour in terms of pourability.

The rate of homogeneous distribution of the granules is determined by the following test. A quantity (0.5 g) of granules are dissolved, with stirring, in one (1) liter of a 50/50% water/acetone mixture and the time (in minutes) necessary to obtain complete disappearance of the granules, observed visually by the experimenter, is measured. For example, a suitable rate of homogeneous distribution of the azo compounds is roughly 5 to 15 minutes.

The term “granules” in the context of the present invention means solid and cohesive agglomerates of constitutive particles, and the particles having a size of roughly between 3 μm and 150 μm, preferably roughly between 10 μm and 150 μm. In general, the granules according to the invention are approximately spherical in shape and the size indicated above corresponds to their diameter. The granules according to the invention can also be in the form of cylinders or other elongated, acicular shapes, where length-to-diameter ratios are roughly between 0.5 and 5, preferably between about 1 and about 3. In the latter case, the size of the granules indicated corresponds to the length of the cylinder.

The cumulative pore volume of the granules can be measured by means of a mercury porosimeter using known techniques.

The composition according to the invention possesses excellent dissolvability in organic solvents and excellent suspendability in water. Moreover, it has a pourability which allows it to be used by means of automatic devices for introducing and feeding solids into reactors. At the same time, it has a content of inhalable particles which is very markedly improved over known pulverulent solid formulations. Finally, its behaviour is satisfactory in terms of not clumping during storage and of being attrition-resistant, in other words its ability not to regenerate fine particles, during storage and transportation.

Among the compounds of formula (I), mention can in particular be made, as examples, of

2,2′-azobis (isobutyronitrile)

2,2′-azobis (2-methylbutyronitrile),

2,2′-azobis (2,4-diinethylvaleronitrile),

2,2′-azobis (2-methylhexylonitrile),

2,2′-azobis (2-cyclopropylpropionitrile),

2,2′-azobis (2-phenylpropionitrile),

1,1′-azobis(1-cyclohexanecarbonitrile) and

4,4′-azobis(4-cyanopentanoic acid).

The compounds of formula (I) in which a group consisting of the combination of R¹, R² is identical to a group consisting of the combination of R³, R⁴ are preferred, in other words compounds which are symmetrical with respect to the plane perpendicular to the —N═N— unit.

According to a preferred embodiment of the invention, the azo compound of formula (I) is 2,2′-azobis (isobutyronitrile), also called AIBN.

The composition according to the invention can comprise, apart from the compound of formula (I) , a surface-active agent. Such compositions are particularly advantageous for their ease of manufacture (short drying time) and their improved content of inhalable particles.

As a surface-active agent that can be used in the composition according to the invention, mention can be made, for example, of salts of the alkylarylsulphonate type, especially alkaline alkylnaphthalenesulphonates, salts of polycarboxylic acids, polycondensates of ethylene oxide and/or propylene oxide with fatty alcohols or with fatty acids or with fatty amines, substituted phenols (especially alkylphenols or arylphenols), salts of sulphosuccinic acid esters, polymers of the arylsulphonate type, especially alkaline polynaphthalenesulphonates obtained by the condensation of (alkyl) arylsulphonates with formaldehyde, lignosulphonates, polyphenylsulphonates, salts of polyacrylic acids, salts of lignosulphonic acids, salts of pheriolsulphonic or naphthalenesulphonic acids, taurine derivatives (especially alkyl taurates), phosphoric esters of polyethoxylated phenols or alcohols, esters of fatty acids and of polyols, derivatives having sulphate, sulphonate and phosphate functional groups of the above compounds and, finally, cationic surfactants such as quaternary ainmoniuin derivatives and pyridinium salts.

Another embodiment of the composition according to the invention, taken alone or in combination with the above embodiment, comprises a disintegrating agent which improves the dispersion of the granules into their constituent particles, after being mixed with the processing liquid. As compounds of this type, mention can be made of bentonites (either natural or activated), starch and its derivatives (especially alkyl starches and carboxyalkyl starches), celluloses (especially microcrystalline cellulose) and cellulose derivatives (especially carboxyalkyl cellulose and methyl cellulose), alginates, soluble inorganic salts, crosslinked polyvinylipyrrolidone, polyethylene glycols, polypropylene glycols and polyvinyl alcohol.

The surface-active agent and the disintegrating agent can be present in the composition according to the invention at a content of roughly between 0.01% and 5%, preferably roughly between 0.05 and 0.5%.

In addition to the constituents described above, the granules of the composition according to the invention can contain antifoaming agents, densifiers, sequestrants, stabilizers, penetrants, preservatives, adhesives, anti-clumping agents, colorants and other additives.

The preferred compositions according to the invention are those which have:

a content of inhalable particles of less than 20 mg, preferably less than 10 mg; and

a flowability of greater than or equal to 4, preferably greater than or equal to 5; and

a rate of homogeneous distribution of less than or equal to 15 minutes, preferably less than or equal to 11 minutes.

The examples below are given by way of illustration of the compositions according to the invention and should in no case be interpreted as limiting the scope of the invention.

### EXAMPLE 1

Granules of AIBN

A ploughshare mixer of the Lödige type (manufactured by the company of the same name) is charged with 100 kg of AIBN powder, obtained after draining at the end of the final step of the synthesis process, having an average size of 50 μm and a 15% moisture content. Using a nozzle, 30 litres of water are sprayed so as to obtain a powder having a 35% moisture content.

This powder is granulated by passing it through a granulating press of the Frewitt type (sold by the company of the same name). The powder is pushed by means of rotating blades against a die consisting of a grid 1 mm in thickness and provided with perforations of 2 mm diameter. During granulation, the temperature of the AIBN powder remains less than 30° C. at every point.

Next, the granules are dried at a temperature of 35° C.

The granules obtained are approximately sphercal and have an average size of 2 mm and a cumulative pore volume of 1.2 ml/g. Their content of inhalable particles is less than 10 mg, their pourability is 5 and their rate of homogeneous distribution is 11 minutes.

### EXAMPLE 2

Example 1 is repeated by spraying the AIBN powder with 15 litres of water in which 100 g of a C₁₃-C₁₅ fatty alcohol condensed with ethylene oxide and with propylene oxide (surfactant) have been dissolved. A powder having a moisture content of 26% is thus obtained.

After granulation and drying, granules whose size is identical to those of Example 1 and whose cumulative pore volume is equal to 1 ml/g are obtained. Their content of inhalable particles is less than 5 mg, their pourability is 4 and their rate of homogeneous distribution is 15 minutes.

### EXAMPLE 3

Example 2 is repeated by adding to the spray water, in addition to the surface-active agent, 100 g of a 4000 molecular weight polyethylene glycol (disintegrating agent).

The granules obtained have the same characteristics, except that their rate of homogeneous distribution is 10 minutes.

We claim:

1. A composition comprising an azo compound of the formula: 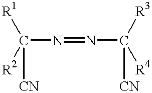
wherein:
R¹, R², R³, and R⁴ each independently represents:
a C₁-C₆-alkyl group, unsubstituted or substituted with a hydroxyl, alkoxy, carboxyl or halogen substituent; or
a C₃-C₆ cycloalkyl group, unsubstituted or substituted with a hydroxyl, alkoxy, carboxyl or halogen substituent;
an aryl group, unsubstituted or substituted with a hydroxyl, alkyl, alkoxy, carboxyl or halogen substituent;
an aralkyl group, unsubstituted or substituted with an alkyl, alkoxy, hydroxyl, carboxyl or halogen substituent; or
at least one of the combinations of R¹ with R² and of R³ with R⁴ forms a cycloalkyl radical; and
wherein the compound is in the form of porous granules having a average size between about 200 μm and about 10 mm and a cumulative pore volume between about 0.2 and about 2 ml per gram of composition.

2. The composition according to claim 1, wherein the average size of the porous granules is between about 250 μm and about 5 mm.

3. The composition according to claim 1, wherein the cumulative pore volume is between about 0.5 and about 1.3 ml per gram of composition.

4. The composition according to claim 1, wherein the compound is:
2,2′-azobis (isobutyronitrile),
2,2′-azobis (2-methylbutyronitrile),
2,2′-azobis (2,4-dimethylvaleronitrile),
2,2′-azobis (2-methylhexylonitrile),
2,2′-azobis (2-cyclopropylpropionitrile),
2,2′-azobis (2-phenylpropionitrile),
1,1′-azobis (1-cyclohexanecarbonitrile) or
4,4′-azobis (4-cyanopentanoic acid).

5. The composition according to claim 1, wherein a group consisting of the combination of R¹, R² is identical to a group consisting of the combination of R³, R⁴.

6. The composition according to claim 4, wherein the compound is 2,2′-azobis (isobutyronitrile).

7. The composition according to claim 1 which further comprises a surface-active agent.

8. The composition according to claim 1 which further comprises a disintegrating agent.

9. The composition according claim 1 which
contains less than about 20 mg inhalable particles among 30 g of granules;
has a flowability of greater than or equal to 4; and
has a rate of homogeneous distribution of less than or equal to about 15 minutes.

10. The composition according to claim 9, which contains less than about 10 mg inhelable particles among 30 g granules.

11. The composition according to claim 9 wherein the flowability is greater than or equal to about 5.

12. The composition according to claim 9, wherein the rate of homogeneous distribution is less than or equal to about 11 minutes.

13. A process for preparing a composition of claim 1, comprising, in succession:
(i) providing an aqueous suspension of the azo compound;
(ii) draining the aqueous suspension to obtain a wet powder containing from about 10 to about 45% of water;
(iii) granulating the wet powder by passing said powder under mechanical stress through a die of thickness e and perforation diameter d, the e/d ratio being between about 0.5 and about 3, the diameter d being approximately the size of the granules, and the stress being such that the temperature of the powder remains at or less than about 40° C.; and
(iv) drying of the granules at a temperature less than about 45° C.

14. The process according to claim 13, wherein the wet powder contains from about 15 to about 45% water.

15. The process according to claim 13, wherein the e/d ratio is between about 0.5 and about 2.

16. The process according to claim 13, wherein the temperature of the powder remains less than about 30° C.

17. The process according to claim 13, wherein the temperature of drying is less than about 35° C.
